(19) United States
(12) Patent Application Publication
(10) Pub. No.: US 2023/0293465 A1
(43) Pub. Date: Sep. 21, 2023

(54) TREATMENT OF LOW-GRADE GLIOMA WITH MIRDAMETINIB

(71) Applicant: St. Jude Children’s Research Hospital, Memphis, TN (US)

(72) Inventors: Suzanne Baker, Memphis, TN (US); Zoran Rankovic, Memphis, TN (US); Burgess Freeman, Memphis, TN (US); Giles Robinson, Memphis, TN (US)

(21) Appl. No.: 18/185,268

(22) Filed: Mar. 16, 2023

Related U.S. Application Data
(60) Provisional application No. 63/321,042, filed on Mar. 17, 2022.

(51) Int. Cl. A61K 31/166; A61K 9/00; A61P 35/00

(52) U.S. Cl. CPC A61K 31/166; A61K 9/0053; A61P 35/00

(57) ABSTRACT

The present disclosure relates to a method of treating a patient (e.g., a human patient) who has low-grade glioma by administering (e.g., orally) mirdametinib, or a pharmaceutically acceptable salt thereof, to the patient.

The present application claims the benefit of U.S. Provisional Application No. 63/321,042, filed Mar. 17, 2022, the entire contents of which is hereby incorporated by reference.

### FIELD OF THE INVENTION

The present disclosure relates to a method of treating a patient (e.g., a human patient) who has low-grade glioma by administering (e.g., orally) mirdametinib, or a pharmaceutically acceptable salt thereof, to the patient.

### BACKGROUND OF THE INVENTION

Low-grade gliomas are brain tumors that originate from glial cells, which support and nourish neurons in the brain. Glial tumors, or gliomas, are divided into four grades, depending on their cells' appearance under a microscope. Grade 1 and 2 gliomas are considered low-grade and account for about two-thirds of all pediatric tumors.

In addition to their grade, low-grade gliomas are also classified based on their location and by the kind of glial cell—astrocytes, oligodendrocytes or ependymocytes—from which they arise.

Low-grade gliomas do not spread outside the brain, but instead grow into the normal brain tissue, creating symptoms as the tumor grows locally. This can disrupt connections between normal brain cells and can also create pressure on the nearby brain. The brain cannot expand when there is a tumor growing within it since it is confined within the skull. As a result, even a relatively small, slow-growing tumor can cause severe brain problems, particularly if the tumor is in a critical area of the brain.

Low-grade gliomas are slow-growing tumors. As they grow, they press on surrounding healthy parts of the brain, affecting their function. As such, the symptoms of a pediatric low-grade glioma depend on the tumor's size and where in the brain it is located. Some of the most common symptoms of a pediatric low-grade glioma may include:
headache, particularly in the morning;
severe or frequent vomiting without other signs of gastrointestinal illness;
vision problems, such as double vision, blurry vision or loss of vision;
difficulty walking or balancing;
seizures;
weight gain or loss;
premature puberty;
clumsiness;
confusion; and
sleepiness.

In many people, a seizure is the first sign of a low-grade glioma. Seizures, which also occur as a result of other conditions, are caused by disorganized electrical activity in the brain. Seizures can cause a loss of consciousness (passing out), involuntary movements (jerking or fits), and/or loss of muscle control throughout the body.

Robinson et al., Oncogene, 2011, 30(11):1341-1350, describes mouse and human glioma cells treated with the MEK inhibitor PD0325901.

There is a need for improved treatments of low-grade glioma. Moreover, objective responses are not sufficient and disease recurrence after completion of therapy is common.

### BRIEF SUMMARY OF THE INVENTION

In one aspect, the present invention relates to a method of treating low-grade glioma in a patient (e.g., a human patient in need thereof) comprising administering (e.g., orally) mirdametinib, or a pharmaceutically acceptable salt thereof, to the patient. In one embodiment, the patient is a pediatric patient.

In another aspect, the present invention relates to a method for treating one or symptoms associated low-grade glioma in a patient (e.g., a human patient in need thereof) comprising administering (e.g., orally) mirdametinib, or a pharmaceutically acceptable salt thereof, to the patient. In certain embodiments, the one or more symptoms associated with low-grade-glioma include, but are not limited to, headache, vomiting, vision problems (such as double vision, blurry vision or loss of vision), difficulty walking or balancing, seizures, weight gain or loss, premature puberty, clumsiness, confusion, sleepiness, and any combination of any of the foregoing. In one embodiment, the patient is a pediatric patient.

In some embodiments of any of the methods described herein, the patient have one or more BRAF, MYB, RAF1, NF1, or FGFR1 mutations (such as a somatic gene rearrangement of BRAF, MYB, RAF1, NF1, or FGFR1).

In some embodiments of any of the methods described herein, the patient has a NF1 germline mutation.

In some embodiments of any of the methods described herein, the patient has metastatic disease.

In some embodiments of any of the methods described herein, a therapeutically effective amount of mirdametinib, or a pharmaceutically acceptable salt thereof, is administered. In some embodiments of any of the methods described herein, the mirdametinib, or a pharmaceutically acceptable salt thereof, is administered in an amount of about 1 mg/m² to about 10 mg/m² per day based on mirdametinib free base.

In some embodiments of any of the methods described herein, the mirdametinib, or a pharmaceutically acceptable salt thereof, is administered in a single dosage form comprising about 0.1 mg/m² to about 10 mg/m² based on mirdametinib free base. In some embodiments of any of the methods described herein, the mirdametinib, or a pharmaceutically acceptable salt thereof, is administered in a single dosage form comprising about 0.1 mg to about 10 mg based on mirdametinib free base.

In some embodiments of any of the methods described herein, the mirdametinib, or a pharmaceutically acceptable salt thereof, is administered once daily. In some embodiments of any of the methods described herein, the mirdametinib, or a pharmaceutically acceptable salt thereof, is administered twice daily.

In one embodiment, 1 mg/m² is orally administered twice daily to the patient. In another embodiment, 2.0 mg/m² is orally administered twice daily to the patient. In yet another embodiment, 2.5 mg/m² is orally administered twice daily to the patient. In yet another embodiment, 3.0 mg/m² is orally administered twice daily to the patient.

In one embodiment of any of the methods described herein,
(a) for a patient having a body surface area between 0.38 m² and 1.80 m², the patient is initially orally administered 3.0 mg/m² mirdametinib twice daily (i.e., a total of 6 mg daily),
(b) for a patient having a body surface area between 0.44 m² and 2.22 m², the patient is initially orally administered 2.0 mg/m² mirdametinib twice daily (i.e., a total of 4 mg daily), and
(c) for a patient having a body surface area between 0.44 m² and 3.0 m², the patient is initially orally administered 1.0 mg/m² mirdametinib twice daily (i.e., a total of 2 mg daily).

In one embodiment of any of the methods described herein,
(a) for a patient having a body surface area no more than 0.69 m², the patient is initially orally administered 1 mg mirdametinib twice daily (i.e., a total of 2 mg daily),
(b) for a patient having a body surface area of 0.7 to 1.04 m², the patient is initially orally administered 2 mg mirdametinib twice daily (i.e., a total of 4 mg daily),
(c) for a patient having a body surface area of 1.05 to 1.49 m², the patient is initially orally administered 3 mg mirdametinib twice daily (i.e., a total of 6 mg daily), and
(d) for a patient having a body surface area of at least 1.5 m², the patient is initially orally administered 4 mg mirdametinib twice daily (i.e., a total of 8 mg daily).

In another embodiment of any of the methods described herein, for a patient 2 to 10 years old and having a body surface area no more than 0.69 m², the patient is initially orally administered 1 mg mirdametinib twice daily (i.e., a total of 2 mg daily). In yet another embodiment, for a patient 2 to 8 years old and having a body surface area no more than 0.69 m², the patient is initially orally administered 1 mg mirdametinib twice daily (i.e., a total of 2 mg daily). In yet another embodiment, for a patient 2 to 7 years old and having a body surface area no more than 0.69 m², the patient is initially orally administered 1 mg mirdametinib twice daily (i.e., a total of 2 mg daily). In yet another embodiment, for a patient 2 to 6 years old and having a body surface area no more than 0.69 m², the patient is initially orally administered 1 mg mirdametinib twice daily (i.e., a total of 2 mg daily). In yet another embodiment, for a patient 2 to 5 years old and having a body surface area no more than 0.69 m², the patient is initially orally administered 1 mg mirdametinib twice daily (i.e., a total of 2 mg daily).

In another embodiment of any of the methods described herein, the dose administered is reduced due to an adverse event, wherein the dose is reduced as follows:
(a) if the dose at the time of the event is 1 mg mirdametinib twice daily, then the reduced daily dose is 1 mg mirdametinib administered in the morning only;
(b) if the dose at the time of the event is 2 mg mirdametinib twice daily, then the reduced daily dose is 2 mg mirdametinib administered in the morning and 1 mg mirdametinib administered in the afternoon or evening;
(c) if the dose at the time of the event is 3 mg mirdametinib twice daily, then the reduced daily dose is 2 mg mirdametinib administered twice daily; and
(d) if the dose at the time of the event is 4 mg mirdametinib twice daily, then the reduced daily dose is 3 mg mirdametinib administered twice daily.

In one embodiment, the adverse event resulting in the dose reduction is acneiform.

In another embodiment of any of the methods described herein, the method comprises orally administering 1 mg mirdametinib twice daily (i.e., a total of 2 mg daily).

In another embodiment of any of the methods described herein, the maximum oral daily dose administered to the patient is 4 mg mirdametinib twice daily (i.e., a total of 8 mg daily).

In one embodiment of any of the methods described herein, about 2 mg/m² mirdametinib is orally administered to the patient twice daily.

In one embodiment of any of the methods described herein, over each four week period, the mirdametinib is administered for the first three weeks and discontinued for the last one week.

In some embodiments of any of the methods described herein, the mirdametinib, or a pharmaceutically acceptable salt thereof, exhibits high blood-brain-barrier penetration.

In some embodiments of any of the methods described herein, the patient is a human. In some embodiments of any of the methods described herein, the human has an age of ≥2 and <25.

In some embodiments of any of the methods described herein, the human has had no prior exposure to MEK inhibitors.

In some embodiments of any of the methods described herein, the mirdametinib, or a pharmaceutically acceptable salt thereof, is administered orally. In some embodiments of any of the methods described herein, the mirdametinib, or a pharmaceutically acceptable salt thereof, is dispersible in a potable liquid or orodispersible in a patient's saliva. In some embodiments of any of the methods described herein, the mirdametinib, or a pharmaceutically acceptable salt thereof, is administered orally as a solid dosage form. In some embodiments of any of the methods described herein, the solid dosage form is a tablet or capsule. In some embodiments of any of the methods described herein, the solid dosage form is a capsule.

In some embodiments of any of the methods described herein, the mirdametinib, or a pharmaceutically acceptable salt thereof, is administered as a monotherapy to treat the low-grade glioma or one or more symptoms associate therewith. In some embodiments of any of the methods described herein, the mirdametinib, or a pharmaceutically acceptable salt thereof, is administered in combination with another active ingredient and/or surgery to treat the low-grade glioma or one or more symptoms associate therewith.

### DETAILED DESCRIPTION OF THE INVENTION

### I. Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs.

In this specification and the appended claims, the singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “mirdametinib” refers to the single enantiomer N-((R)-2,3-dihydroxypropoxy)-3,4-difluoro-2-(2-fluoro-4-iodo-phenylamino)benzamide. Mirdametinib is an allosteric, small molecule targeting mitogen-activated protein kinase kinase (MEK). The teachings throughout the specification regarding mirdametinib equally apply to pharmaceutically acceptable salts of mirdametinib.

The term “mg/m²” refers to the dose in milligrams per m² body surface area of the patient.

The term “subject” refers to an animal, including, but not limited to, a primate (e.g., human), cow, sheep, goat, horse, dog, cat, rabbit, rat, or mouse. The terms “subject” and “patient” are used interchangeably herein in reference, for example, to a mammalian subject, such as a human subject.

The term “pediatric” refers to a human subject under the age of 21 years at the time of treatment. The term “pediatric” can be further divided into various subpopulations including: neonates (from birth through the first 28 days of life); infants (29 days of age to less than two years of age); young children (two years of age to less than 12 years of age); and adolescents (12 years of age through 21 years of age (up to, but not including, the twenty-second birthday)). See, e.g., Berhman et al., Textbook of Pediatrics, 15th Ed. Philadelphia: W.B. Saunders Company, 1996; Rudolp et al., Rudolph's Pediatrics, 21st Ed. New York: McGraw-Hill, 2002; and Avery et al., Pediatric Medicine, 2nd Ed. Baltimore: Williams & Wilkins; 1994. In some additional embodiments, a pediatric patient is 2 to 21 years of age, 3 to 21 years of age, 2 to 16 years of age, 2 to 15 years of age, 2 to 8 years of age, 2 to 7 years of age, 2 to 6 years of age or 2 to 5 years of age. Younger pediatric patients in particular, such as neonates, infants and young children, can have difficulty swallowing whole capsules or tablets.

The term “dispersible” as used herein refers to a composition (e.g., a tablet, powder, granules, minitablets, or pellets) which disintegrates and/or dissolves when combined with water or another potable liquid (e.g., a non-water beverage), or a subject's own saliva when placed in the subject's mouth, with or without the addition of agitation or temperature modification. In some embodiments, the dispersible composition disintegrates or dissolves within 10 minutes, 9 minutes, 8 minutes, 7 minutes, 6 minutes, 5 minutes, 4 minutes, 3 minutes, 2 minutes, or 1 minute after being combined with water or another potable liquid. Such disintegration or dissolution need not be complete. For example, a dispersible tablet may dissolve almost entirely, but some undissolved particulate matter may remain.

The term “orodispersible” refers to a composition which is capable of dissolving or disintegrating in a subject's mouth (i.e., dissolving or disintegrating in a subject's saliva) if administered orally, without a requirement of first dissolving or disintegrating in a separate container.

As used herein, the terms “treat,” “treated,” and “treating” mean both therapeutic treatment and prophylactic or preventative measures wherein the object is to prevent or slow down (lessen) an undesired physiological condition, disorder, or disease, or obtain beneficial or desired clinical results. Thus, those in need of treatment include those already diagnosed with or suspected of having the disorder. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment. The term “therapeutically effective amount” is meant to include the amount of a compound that, when administered, is sufficient to prevent development of, or alleviate to some extent, one or more of the symptoms of a disorder, disease, or condition being treated. The term “therapeutically effective amount” also refers to the amount of a compound that is sufficient to elicit the biological or medical response of a cell, tissue, system, animal, or human, which is being sought by a researcher, veterinarian, medical doctor, or clinician.

In certain aspects, a subject is successfully “treated” for a tumor, according to the methods described herein if the patient shows one or more of the following: a reduction in the size of the tumor; relief of one or more symptoms associated with the specific tumor; a reduction in the volume of the tumor; improvement in quality of life; increased progression-free survival (PFS), disease-free survival (DFS), overall survival (OS), metastasis-free survival (MFS), complete response (CR), minimal residual disease (MRD), partial response (PR), stable disease (SD), a decrease in progressive disease (PD), an increased time to progression (TTP), or any combination thereof. In some aspects, nationally or internationally accepted standards of treatment outcomes in a given tumor can be used to determine whether an effective amount of mirdametinib meets any of these particular endpoints (e.g., CR, PFS, PR).

In certain aspects, a subject is successfully “treated” for cancer, e.g., low-grade glioma, according to the methods described herein if the patient shows one or more of the following: a reduction in the size of the tumor; relief of one or more symptoms associated with the specific tumor; a reduction in the number of or complete absence of cancer cells; relief of one or more symptoms associated with the specific cancer; reduced morbidity and mortality; improvement in quality of life; increased progression-free survival (PFS), disease-free survival (DFS), overall survival (OS), metastasis-free survival (MFS), complete response (CR), minimal residual disease (MRD), partial response (PR), stable disease (SD), a decrease in progressive disease (PD), an increased time to progression (TTP), or any combination thereof. In some embodiments, nationally or internationally accepted standards of treatment outcomes in a given cancer can be used to determine whether an effective amount of mirdametinib meets any of these particular endpoints (e.g., CR, PFS, PR).

The terms “pharmaceutically acceptable carrier,” “pharmaceutically acceptable excipient,” “physiologically acceptable carrier,” or “physiologically acceptable excipient” refer to a pharmaceutically acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, Gibson Ed., CRC Press LLC: Boca Raton, FL, 2004 (incorporated herein by reference).

The term “pharmaceutically acceptable salts” refers to the relatively non-toxic, inorganic and organic acid addition salts of mirdametinib. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include, but are not limited to, the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts. See, e.g., Berge et al., “Pharmaceutical Salts”, J. Pharm. Sci., 66, 1-19, 1977.

The pharmaceutically acceptable salts of mirdametinib also include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, and nitric acid; and the salts prepared from organic acids such as, but not limited to, acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, and isothionic.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

### II. Methods of Treatment

Methods for treating low-grade glioma, comprising administering to a patient (e.g., a human patient in need thereof) mirdametinib or a pharmaceutically acceptable salt thereof are provided herein.

Methods for treating one or more symptoms associate with low-grade glioma comprising administering to a patient (e.g., a patient in need thereof) mirdametinib, or a pharmaceutically acceptable salt thereof, are provided herein

In some embodiments of any of the methods described herein, a therapeutically effective amount of mirdametinib, or a pharmaceutically acceptable salt thereof, is administered.

In some embodiments of any of the methods described herein, the mirdametinib, or a pharmaceutically acceptable salt thereof, is administered in an amount of about 1 mg/m² to about 10 mg/m² per day based on mirdametinib free base, about 1.5 mg/m² to about 9.5 mg/m² per day based on mirdametinib free base, about 2 mg/m² to about 9 mg/m² per day based on mirdametinib free base, about 2.5 mg/m² to about 8.5 mg/m² per day based on mirdametinib free base, about 3 mg/m² to about 8 mg/m² per day based on mirdametinib free base, about 3.5 mg/m² to about 7.5 mg/m² per day based on mirdametinib free base, about 4 mg/m² to about 7 mg/m² per day based on mirdametinib free base, about 4.5 mg/m² to about 6.5 mg/m² per day based on mirdametinib free base, or about 5 mg/m² to about 6 mg/m² per day based on mirdametinib free base. In some embodiments of any of the methods described herein, the mirdametinib, or a pharmaceutically acceptable salt thereof, is administered in an amount of about 1 mg/m² per day based on mirdametinib free base, about 1.5 mg/m² per day based on mirdametinib free base, about 2 mg/m² per day based on mirdametinib free base, about 2.5 mg/m² per day based on mirdametinib free base, about 3 mg/m² per day based on mirdametinib free base, about 3.5 mg/m² per day based on mirdametinib free base, about 4 mg/m² per day based on mirdametinib free base, about 4.5 mg/m² per day based on mirdametinib free base, about 5 mg/m² per day based on mirdametinib free base, about 5.5 mg/m² per day based on mirdametinib free base, about 6 mg/m² per day based on mirdametinib free base, about 6.5 mg/m² per day based on mirdametinib free base, about 7 mg/m² per day based on mirdametinib free base, about 7.5 mg/m² per day based on mirdametinib free base, about 8 mg/m² per day based on mirdametinib free base, about 8.5 mg/m² per day based on mirdametinib free base, about 9 mg/m² per day based on mirdametinib free base, about 9.5 mg/m² per day based on mirdametinib free base, or about 10 mg/m² per day based on mirdametinib free base.

In some embodiments of any of the methods described herein, the mirdametinib, or a pharmaceutically acceptable salt thereof, is administered in a single dosage form comprising about 0.1 mg/m² to about 10 mg/m² based on mirdametinib free base, about 0.5 mg/m² to about 9.5 mg/m² based on mirdametinib free base, about 1 mg/m² to about 9 mg/m² based on mirdametinib free base, about 1.5 mg/m² to about 8.5 mg/m² based on mirdametinib free base, about 2 mg/m² to about 8 mg/m² based on mirdametinib free base, about 2.5 mg/m² to about 7.5 mg/m² based on mirdametinib free base, about 3 mg/m² to about 7 mg/m² based on mirdametinib free base, about 3.5 mg/m² to about 6.5 mg/m² based on mirdametinib free base, about 4 mg/m² to about 6 mg/m² based on mirdametinib free base, or about 4.5 mg/m² to about 5.5 mg/m² based on mirdametinib free base. In some embodiments of any of the methods described herein, the mirdametinib, or a pharmaceutically acceptable salt thereof, is administered in a single dosage form comprising about 0.1 mg/m² based on mirdametinib free base, about 0.2 mg/m² based on mirdametinib free base, about 0.3 mg/m² based on mirdametinib free base, about 0.4 mg/m² based on mirdametinib free base, about 0.5 mg/m² based on mirdametinib free base, about 1 mg/m² based on mirdametinib free base, about 1.5 mg/m² based on mirdametinib free base, about 2 mg/m² based on mirdametinib free base, about 2.5 mg/m² based on mirdametinib free base, about 3 mg/m² based on mirdametinib free base, about 3.5 mg/m² based on mirdametinib free base, about 4 mg/m² based on mirdametinib free base, about 4.5 mg/m² based on mirdametinib free base, about 5 mg/m² based on mirdametinib free base, about 5.5 mg/m² based on mirdametinib free base, about 6 mg/m² based on mirdametinib free base, about 6.5 mg/m² based on mirdametinib free base, about 7 mg/m² based on mirdametinib free base, about 7.5 mg/m² based on mirdametinib free base, about 8 mg/m² based on mirdametinib free base, about 8.5 mg/m² based on mirdametinib free base, about 9 mg/m² based on mirdametinib free base, about 9.5 mg/m² based on mirdametinib free base, or about 10 mg/m² based on mirdametinib free base.

In some embodiments of any of the methods described herein, the mirdametinib, or a pharmaceutically acceptable salt thereof, is administered one, two, three, or four times per day. In some embodiments of any of the methods described herein, the mirdametinib, or a pharmaceutically acceptable salt thereof, is administered once daily. In some embodiments of any of the methods described herein, the mirdametinib, or a pharmaceutically acceptable salt thereof, is administered twice daily.

In some embodiments of any of the methods described herein , the mirdametinib, or a pharmaceutically acceptable salt thereof, is administered twice daily in an amount of about 0.5 mg/m² to about 10 mg/m² based on mirdametinib free base, about 1 mg/m² to about 9.5 mg/m² based on mirdametinib free base, about 1.5 mg/m² to about 9 mg/m² based on mirdametinib free base, about 2 mg/m² to about 8.5 mg/m² based on mirdametinib free base, about 2.5 mg/m² to about 8 mg/m² based on mirdametinib free base, about 3 mg/m² to about 7.5 mg/m² based on mirdametinib free base, about 3.5 mg/m² to about 7 mg/m² based on mirdametinib free base, about 4 mg/m² to about 6.5 mg/m² based on mirdametinib free base, about 4.5 mg/m² to about 6 mg/m² based on mirdametinib free base, or about 5 mg/m² to about 6 mg/m² based on mirdametinib free base. In some embodiments of any of the methods described herein v, the mirdametinib, or a pharmaceutically acceptable salt thereof, is administered twice daily in an amount of about 0.5 mg/m² based on mirdametinib free base, about 1 mg/m² based on mirdametinib free base, about 1.5 mg/m² based on mirdametinib free base, about 2 mg/m² based on mirdametinib free base, about 2.5 mg/m² based on mirdametinib free base, about 3 mg/m² based on mirdametinib free base, about 3.5 mg/m² based on mirdametinib free base, about 4 mg/m² based on mirdametinib free base, about 4.5 mg/m² based on mirdametinib free base, about 5 mg/m² based on mirdametinib free base, about 5.5 mg/m² based on mirdametinib free base, about 6 mg/m² based on mirdametinib free base, about 6.5 mg/m² based on mirdametinib free base, about 7 mg/m² based on mirdametinib free base, about 7.5 mg/m² based on mirdametinib free base, about 8 mg/m² based on mirdametinib free base, about 8.5 mg/m² based on mirdametinib free base, about 9 mg/m² based on mirdametinib free base, about 9.5 mg/m² based on mirdametinib free base, or about 10 mg/m² based on mirdametinib free base.

In some embodiments of any of the methods described herein , the mirdametinib, or a pharmaceutically acceptable salt thereof, is administered as mirdametinib free base.

In one aspect, the present invention relates to a method for treating low-grade glioma in a patient (e.g., a patient in need thereof) comprising administering mirdametinib free base to the patient.

In some embodiments of any of the methods described herein, a therapeutically effective amount of mirdametinib free base is administered.

In some embodiments of any of the methods described herein, the mirdametinib free base is administered in an amount of about 1 mg/m² to about 10 mg/m² per day, about 1.5 mg/m² to about 9.5 mg/m² per day, about 2 mg/m² to about 9 mg/m² per day, about 2.5 mg/m² to about 8.5 mg/m² per day, about 3 mg/m² to about 8 mg/m² per day, about 3.5 mg/m² to about 7.5 mg/m² per day, about 4 mg/m² to about 7 mg/m² per day, about 4.5 mg/m² to about 6.5 mg/m² per day, or about 5 mg/m² to about 6 mg/m² per day. In some embodiments of any of the methods described herein, the mirdametinib free base is administered in an amount of about 1 mg/m² per day, about 1.5 mg/m² per day, about 2 mg/m² per day, about 2.5 mg/m² per day, about 3 mg/m² per day, about 3.5 mg/m² per day, about 4 mg/m² per day, about 4.5 mg/m² per day, about 5 mg/m² per day, about 5.5 mg/m² per day, about 6 mg/m² per day, about 6.5 mg/m² per day, about 7 mg/m² per day, about 7.5 mg/m² per day, about 8 mg/m² per day, about 8.5 mg/m² per day, about 9 mg/m² per day, about 9.5 mg/m² per day, or about 10 mg/m² per day.

In some embodiments of any of the methods described herein, the mirdametinib free base is administered in a single dosage form comprising about 0.1 mg/m² to about 10 mg/m², about 0.5 mg/m² to about 9.5 mg/m², about 1 mg/m² to about 9 mg/m², about 1.5 mg/m² to about 8.5 mg/m², about 2 mg/m² to about 8 mg/m², about 2.5 mg/m² to about 7.5 mg/m², about 3 mg/m² to about 7 mg/m², about 3.5 mg/m² to about 6.5 mg/m², about 4 mg/m² to about 6 mg/m², or about 4.5 mg/m² to about 5.5 mg/m². In some embodiments of any of the methods described herein, the mirdametinib free base is administered in a single dosage form comprising about 0.1 mg/m², about 0.2 mg/m², about 0.3 mg/m², about 0.4 mg/m², about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m^{2.}

In some embodiments of any of the methods described herein, the mirdametinib free base is administered one, two, three, or four times per day. In some embodiments of any of the methods described herein, the mirdametinib free base is administered once daily. In some embodiments of any of the methods described herein, the mirdametinib free base is administered twice daily.

In some embodiments of any of the methods described herein, the mirdametinib free base is administered twice daily in an amount of about 0.5 mg/m² to about 10 mg/m², about 1 mg/m² to about 9.5 mg/m², about 1.5 mg/m² to about 9 mg/m², about 2 mg/m² to about 8.5 mg/m², about 2.5 mg/m² to about 8 mg/m², about 3 mg/m² to about 7.5 mg/m², about 3.5 mg/m² to about 7 mg/m², about 4 mg/m² to about 6.5 mg/m², about 4.5 mg/m² to about 6 mg/m², or about 5 mg/m² to about 6 mg/m². In some embodiments of any of the methods described herein, the mirdametinib free base is administered twice daily in an amount of about 0.5 mg/m², about 1 mg/m², about 1.5 mg/m², about 2 mg/m², about 2.5 mg/m², about 3 mg/m², about 3.5 mg/m², about 4 mg/m², about 4.5 mg/m², about 5 mg/m², about 5.5 mg/m², about 6 mg/m², about 6.5 mg/m², about 7 mg/m², about 7.5 mg/m², about 8 mg/m², about 8.5 mg/m², about 9 mg/m², about 9.5 mg/m², or about 10 mg/m^{2.}

In some embodiments of any of the methods described herein, the mirdametinib, or a pharmaceutically acceptable salt thereof, exhibits high blood-brain-barrier penetration.

In some embodiments of any of the methods described herein, the patient is a human.

In some embodiments of any of the methods described herein, the human has an age of ≥2 and <25.

In some embodiments of any of the methods described herein, the human has no prior exposure to MEK inhibitors. In some embodiments of any of the methods described herein, the human has not responded to prior treatment to one or more MEK inhibitors.

In some embodiments of any of the methods described herein, the mirdametinib, or a pharmaceutically acceptable salt thereof, is administered orally. In some embodiments of any of the methods described herein, the mirdametinib, or a pharmaceutically acceptable salt thereof, is administered orally as a solid dosage form. In some embodiments of any of the methods described herein, the solid dosage form is a tablet or capsule. In some aspects, the solid dosage form is a capsule. In some embodiments of any of the methods described herein, the mirdametinib, or a pharmaceutically acceptable salt thereof, is dispersible in a potable liquid or orodispersible in a patient's saliva.

In some embodiments of any of the methods described herein, the mirdametinib, or a pharmaceutically acceptable salt thereof, is administered as a monotherapy to treat the low-grade glioma or a symptom thereof.

In some embodiments of any of the methods described herein, the mirdametinib, or a pharmaceutically acceptable salt thereof, is administered in combination with another active ingredient and/or surgery to treat the low-grade glioma or a symptom thereof.

### EXAMPLE

### Example 1: Phase I/II Evaluation of Single Agent Mirdametinib, a Brain-Penetrant MEK1/2 Inhibitor, for the Treatment of Children, Adolescents, and Young Adults with Low-Grade Glioma (LGG)

A multi-arm phase I/II trial of mirdametinib is currently being conducted in patients ≥2 and <25 years with LGG. Phase I requires participants have no prior exposure to MEK inhibitors and recurrent/progressive disease with biopsy-proven evidence of MAPK pathway activation. Three escalating dose levels (2 mg/m²/dose BID, 2.5 mg/m²/dose BID and 3 mg/m²/dose BID) are planned using a rolling 6 design.

The median age enrolled in the study is 10 years (3-21 years old). 10 patients have somatic gene rearrangements (7 BRAF, 1 MYB, 1 RAF1, 1 FGFR1); 1 patient has a NF1 germline mutation; and 3 patients have metastatic disease. No dose-limiting toxicities occurred for dose level 1 (2 mg/m²) whereas data are pending for dose level 2 (2.5 mg/m²). Only grade 1 and 2 treatment-related adverse events have been observed. No MEK related retinopathy or cardiopathy has been observed. Four of the six patients who completed at least one follow-up disease evaluation have a minor response (>25% -<50% decrease). No disease progressions have occurred.

Of the 11 patients having a somatic gene rearrangement or NF1, 4 patients with BRAF and the 1 patient with FGFR1 received a dose of 2 mg/m² BID. The remainder of the 11 patients (3 BRAF, 1 MYB, 1 NF1, and 1 RAF1) received a dose of 2.5 mg/m² BID. The results as of a particular cut-off date are shown in Table 1 below.

TABLE 1

| | 2 mg/m² BID | 2.5 mg/m² BID | Total |
|---|---|---|---|
| | (N = 5) | (N = 6) | (N = 11) |
| Partial Response (PR) | 1 (20.0%) | | 1 (9.1%) |
| Minor Response (MR) | 4 (80.0%) | 3 (50.0%) | 7 (63.6%) |
| Stable Disease (SD) | | 3 (50.0%) | 3 (27.3%) |

PR = decrease in tumor size by 50-75%;
MR = decrease by 25-50%;
SD - stable tumor size or decrease up to 25%.

Thus far, mirdametinib is well-tolerated and clinically promising when dosed continuously in patients with recurrent/progressive pediatric LGG (pLGG).

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

1. A method of treating low-grade glioma in a patient in need thereof comprising administering mirdametinib or a pharmaceutically acceptable salt thereof to the patient.

2. The method of claim 1, wherein the patient has one or more BRAF, MYB, RAF 1, or FGFR1 mutations.

3. The method of claim 1, wherein a therapeutically effective amount of mirdametinib, or a pharmaceutically acceptable salt thereof, is administered.

4. The method of claim 1, wherein the mirdametinib, or a pharmaceutically acceptable salt thereof, is orally administered in an amount of about 1 mg/m² to about 10 mg/m² per day based on mirdametinib free base.

5. The method of claim 1, wherein the mirdametinib, or a pharmaceutically acceptable salt thereof, is orally administered in an amount of about 4 mg/m² per day based on mirdametinib free base.

6. The method of claim 1, wherein the mirdametinib, or a pharmaceutically acceptable salt thereof, is orally administered in an amount of about 5 mg/m² per day based on mirdametinib free base.

7. The method of claim 1, wherein the mirdametinib, or a pharmaceutically acceptable salt thereof, is orally administered in an amount of about 6 mg/m² per day based on mirdametinib free base.

8. The method of claim 1, wherein the mirdametinib, or a pharmaceutically acceptable salt thereof, is orally administered once daily.

9. The method of claim 1, wherein the mirdametinib, or a pharmaceutically acceptable salt thereof, is orally administered twice daily.

10. The method of claim 1, wherein the mirdametinib, or a pharmaceutically acceptable salt thereof, is orally administered twice daily in an amount of about 2 mg/m² based on mirdametinib free base.

11. The method of claim 1, wherein the mirdametinib, or a pharmaceutically acceptable salt thereof, is orally administered twice daily in an amount of about 2.5 mg/m² based on mirdametinib free base.

12. The method of claim 1, wherein the mirdametinib, or a pharmaceutically acceptable salt thereof, is orally administered twice daily in an amount of about 3 mg/m² based on mirdametinib free base.

13. The method of claim 1, wherein
(a) for a patient having a body surface area no more than 0.69 m², the patient is initially orally administered 1 mg mirdametinib twice daily,
(b) for a patient having a body surface area of 0.7 to 1.04 m², the patient is initially orally administered 2 mg mirdametinib twice daily,
(c) for a patient having a body surface area of 1.05 to 1.49 m², the patient is initially orally administered 3 mg mirdametinib twice daily, and
(d) for a patient having a body surface area of at least 1.5 m², the patient is initially orally administered 4 mg mirdametinib twice daily.

14. The method of claim 1 any one of claims 1 3, wherein
(a) for a patient having a body surface area between 0.38 m² and 1.80 m², the patient is initially orally administered 3.0 mg/m² mirdametinib twice daily,
(b) for a patient having a body surface area between 0.44 m² and 2.22 m², the patient is initially orally administered 2.0 mg/m² mirdametinib twice daily, and
(c) for a patient having a body surface area between 0.44 m² and 3.0 m², the patient is initially orally administered 1.0 mg/m² mirdametinib twice daily.

15. The method of claim 1 any one of the preceding claims, wherein the dose administered is reduced due to an adverse event, wherein the dose is reduced as follows:
(a) if the dose at the time of the event is 1 mg mirdametinib twice daily, then the reduced daily dose is 1 mg mirdametinib orally administered in the morning only;
(b) if the dose at the time of the event is 2 mg mirdametinib twice daily, then the reduced daily dose is 2 mg mirdametinib orally administered in the morning and 1 mg mirdametinib administered in the afternoon or evening;
(c) if the dose at the time of the event is 3 mg mirdametinib twice daily, then the reduced daily dose is 2 mg mirdametinib orally administered twice daily; and
(d) if the dose at the time of the event is 4 mg mirdametinib twice daily, then the reduced daily dose is 3 mg mirdametinib orally administered twice daily.

16. The method of claim 15, wherein the adverse event resulting in the dose reduction is acneiform.

17. The method of claim 1, wherein over each four week period, the mirdametinib is administered for the first three weeks and discontinued for the last one week.

18. The method of claim 1, wherein the patient has an age of ≥2 and <25.

19. The method of claim 1, wherein the patient is 2 to 21 years of age.

20. The method of claim 1, wherein the patient is a pediatric patient.

21. The method of claim 1, wherein the patient is 2 to 15 years of age.

22. The method of claim 1, wherein the human has no prior exposure to MEK inhibitors.

23. The method of claim 1, wherein the mirdametinib, or a pharmaceutically acceptable salt thereof, is administered as a monotherapy to treat the low-grade glioma.

24. The method of claim 1, wherein the mirdametinib, or a pharmaceutically acceptable salt thereof, is administered in combination with another active ingredient and/or surgery to treat the low-grade glioma.
